(19) United States
(12) Patent Application Publication
(10) Pub. No.: US 2010/0228204 A1
(43) Pub. Date: Sep. 9, 2010

(54) FACIAL TREATMENT MASK COMPRISING AN ISOLATION LAYER

(76) Inventors: Heidi Beatty, Old Tappan, NJ (US); Dan Formosa, Piermont, NY (US); Julie Francoeur, Boumemouth (GB); Sachiko Uozumi, New York, NY (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 12/696,674

(22) Filed: Jan. 29, 2010

Related U.S. Application Data
(60) Provisional application No. 61/148,213, filed on Jan. 29, 2009.

(51) Int. Cl. A61M 35/00; A42B 1/18

(52) U.S. Cl. 604/303; 2/206

(57) ABSTRACT

The present invention features a facial mask including a water-insoluble, liquid-retaining layer sized and shaped to lie against and substantially coincident with a face of a human user and having at least one opening formed therethrough. The facial mask also includes an isolation layer substantially overlapping the water-insoluble, liquid-retaining layer. The isolation layer is readily separable from said water-insoluble, liquid-retaining layer. At least one opening in the water-insoluble, liquid-retaining layer is unobstructed by the isolation layer.

### FIELD OF THE INVENTION

The present invention relates to a facial mask and uses thereof to treat skin.

### BACKGROUND OF THE INVENTION

Products such as cleansers and moisturizers formulated with vitamins and other skin benefit agents have been used for many years to treat the skin. Employing a water-insoluble substrate such as a facial mask to assist in the process of cleansing, moisturizing and delivery of certain benefit agents to the skin is also known. For example, consumers typically use hydrating facial mask products for treatment of various skin conditions as well as to improve the physical appearance and texture of the facial skin. This can be accomplished while the user relaxes, such as in a prone position, while the mask contacts the skin of the face, and provides benefits thereto.

However, in practice conventional facial masks are difficult to apply to the face. Since a facial mask needs to be large enough to contact the entire face but conserve space on the store shelf, the facial mask generally must be sold in a folded state. As such, the product must be unfolded and properly oriented to align the eyeholes, nose slits and the like with the corresponding facial features of the user. For user convenience, typical facial masks are sold saturated with a liquid impregnate. However, the presence of liquid impregnate magnifies the difficulty in unfolding the facial mask, since a wet facial mask tends to adhere to itself. This tends to frustrate the user's attempts to unfold and deploy the facial mask. The problem is further exacerbated since often the mask comprises only a thin fibrous layer, which makes it even more difficult to peel apart from itself.

It is known in the art to “sandwich” a hydrogel-type liquid-retaining layer of a facial mask between two separate isolation layers contacting either surface of the liquid-retaining layer. This configuration has particular drawbacks. First, this configuration utilizes an excessive amount of material, since both sides of the liquid-retaining layer are covered by a separate isolation layer. Second, the isolation layers have no holes, so that when the user removes one isolation layer and places the liquid-retaining layer against her face, the remaining isolation layer is uncomfortably positioned against her eyes and mouth.

Applicants have now developed a facial mask that is much more comfortable to apply to the face, saves on material costs, and surprisingly is still easy to unfold and deploy onto the face. The facial mask comprises a water-insoluble, liquid-retaining layer sized and shaped to lie against and substantially coincident with a face of a human user and an isolation layer substantially overlapping the liquid-retaining layer. The water-insoluble, liquid-retaining layer comprises one or more openings. Furthermore, at least one opening of the liquid-retaining layer is unobstructed by the isolation layer. In use, the isolation layer is readily separated from the liquid-retaining layer.

### BRIEF DESCRIPTION OF THE DRAWINGS

FIG. 1 is a top plan view of a liquid-retaining layer of a facial mask in accordance with a first embodiment of the present invention;

FIG. 2 is a top perspective view of the liquid-retaining layer of FIG. 1 and an isolation layer for use therewith;

FIG. 3 is a top perspective view of the liquid-retaining layer of FIG. 2 contacting the isolation layer;

FIG. 4 is top perspective view of the liquid-retaining layer and the isolation layer of FIG. 3 partially folded along common fold axes;

FIG. 5 is a top perspective view of the liquid-retaining layer and the isolation layer of FIG. 3 in a more completely folded state;

FIG. 6 is a top perspective view of the liquid-retaining layer and the isolation layer of FIG. 3 folded along an additional common fold axis;

FIG. 7 is a top perspective view of the liquid-retaining layer and the isolation layer of FIG. 3 co-folded and suitable for placement into a container;

FIG. 8 is a top plan view of an alternative embodiment of the inventive facial mask, wherein the facial mask includes a T-shaped isolation layer;

FIG. 9 is a top perspective view of an apparatus that may be used to test adherence of an isolation layer to a liquid-retaining layer.

### SUMMARY OF THE INVENTION

In one aspect of the invention, a facial mask comprises a water-insoluble, liquid-retaining layer sized and shaped to lie against and substantially coincident with a face of a human user. The water-insoluble, liquid-retaining layer comprises at least one opening formed therethrough. The facial mask further comprises an isolation layer substantially overlapping said water-insoluble, liquid-retaining layer. The opening in the water-insoluble, liquid-retaining layer is unobstructed by the isolation layer. In use, the isolation layer is readily separated from said water-insoluble, liquid-retaining layer.

### DETAILED DESCRIPTION OF THE INVENTION

It is believed that one of ordinary skill in the art can, based upon the description herein, utilize the present invention to its fullest extent. The following specific embodiments of the invention are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs. Whenever used, any percentage is weight by weight (% w/w) unless otherwise indicated.

The present invention is directed to a facial mask including a liquid-retaining layer sized and shaped to lie against and substantially coincident with the face of a human user. The facial mask further includes an isolation layer substantially overlapping the liquid-retaining layer. According to the invention, at least one opening formed in the liquid-retaining layer is unobstructed by the isolation layer.

In use, the facial mask may be readily unfolded, and the liquid-retaining layer may then be placed comfortably against a surface of the skin with the isolating layer on the opposite surface of the liquid-retaining layer, facing outward. Advantageously, the openings in the liquid-retaining layer remain unobstructed by the isolation layer. The isolation layer may be readily separated from the liquid-retaining layer and discarded, leaving the liquid-retaining layer in contact with the user's skin.

### Liquid-Retaining Layer (Liquid-Retaining Layer)

The facial mask of the present invention comprises a liquid-retaining layer. The term “layer,” as used in this specification means an expanse of material having a length and/or width substantially larger (such as 5 to 10 or more times larger) than its thickness. The liquid-retaining layer is generally water-insoluble. As used herein, “water-insoluble” means the failure to readily dissolve or break apart upon immersion in distilled water at 25° C. While portions of the liquid-retaining layer may be leachable or readily soluble in distilled water, at least one portion of the liquid-retaining layer remains intact. The intact portion may be readily manipulated, such as picked up and transported as an interconnected cohesive unit, by a user.

The liquid-retaining layer is capable or retaining a liquid impregnate (such as by absorbing the liquid impregnate among, along, and/or between fibers that comprise the liquid-retaining layer) for a period of time at least as long as from when the product is manufactured to a time when the product is used by a consumer (i.e., a shelf storage period). In this embodiment of the invention, during this shelf storage period the liquid-retaining layer should generally maintain its mechanical integrity such that a user can lay the liquid-retaining layer onto the skin and transfer liquid impregnate thereto. Furthermore, the liquid-retaining layer is generally capable of holding portions of liquid impregnate against the facial skin when used.

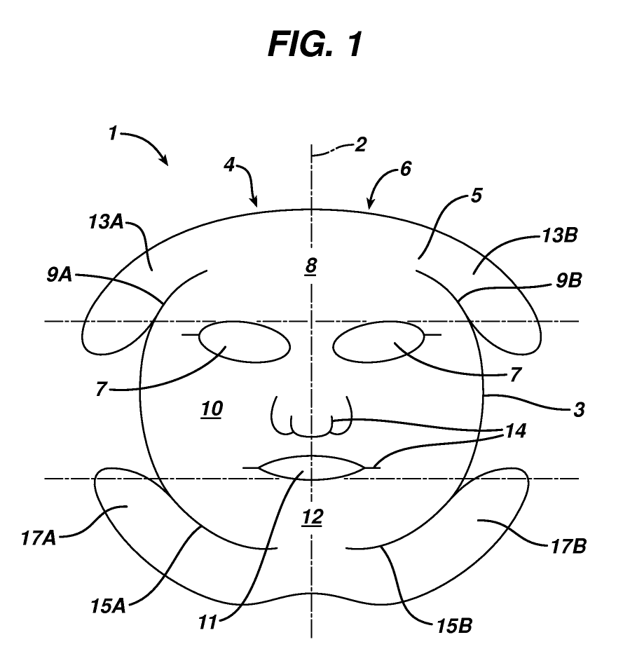

FIG. 1 depicts one embodiment of a facial mask consistent with embodiments of the invention described herein. Facial mask 1 includes a liquid-retaining layer 5 that is generally sized and shaped to lie against the face of a user. It is preferred that the liquid-retaining layer 5 lie substantially flat against the face of the user, i.e., the liquid-retaining layer 5 is capable of draping across the face and generally conforming to the curvature of the face. Preferably, there are no substantial gaps between the face and the liquid retaining layer 5. Such uniform contact between the liquid retaining layer 5 and the face of the user generally allows a liquid impregnate present in the liquid retaining layer 5 to contact the entire face of the user. The liquid-retaining layer 5 is generally further capable of lying substantially coincident with the face of the user, i.e., it generally only requires simple manipulation such as unfolding or at most slight tearing of preformed perforations in order to assume a shape that coincides with a human face. In a preferred embodiment, the facial mask is sized and shaped to lie substantially flatly against the entire face of a user. As used herein, “entire face” means the majority, e.g., at least about 90%, of the face, including the nose, cheeks, around the eyes, and the forehead, as well as under and above the mouth.

The liquid-retaining layer 5 may further comprise a vertical centerline, preferably a vertical axis of symmetry 2 that separates a left side 4 from a right side 6 of the facial mask 1. Furthermore, the liquid-retaining layer 5 comprises a perimeter 3 that defines the outer boundary of the facial mask when it is laid flat such as on a tabletop.

The liquid-retaining layer 5 includes, within the perimeter 3, at least one opening for the eyes, mouth or nose. The opening may be pre-cut, in which case it will be visible as area devoid of liquid-retaining layer. Alternatively, the opening may be defined by a perforated or weakened line. In this embodiment, the user can readily separate, using little force and in a controlled fashion, the liquid-retaining layer along the perforated or weakened line. For example, a user may “punch” or “tear” the liquid-retaining layer along such a line or other shape in a controlled fashion prior to use.

As shown in FIG. 1, the liquid-retaining layer 5 includes pre-cut openings 7 for the eyes of a user. It is further desirable that facial mask 1 include a pre-cut opening 11 for the mouth of the user.

The liquid-retaining layer 5 may further include at least one interior slit. As used herein, an “interior slit” is a pre-cut line in the liquid-retaining layer within but not touching the perimeter. Interior slits are formed by slicing the liquid-retaining layer when laid flat and are often visible only as lines or boundaries when the liquid-retaining layer 5 is laid flat. For example, facial mask 1 includes one or more interior slits 14 that permit the nostril area or the mouth of the user to be exposed (for instance, uncovered) in use.

Interior slits 14 may be straight or curved. In one embodiment, in order to provide better fit, one or more interior slits 14 for the nose may include a plurality of arcuate portions to facilitate adherence to an underside of the user's nasal ridge, rather than, in the case of a user with a small nose, dangling or hanging off of the nose.

The liquid-retaining layer 5 includes a forehead region 8 comprising that portion of the liquid-retaining layer 5 entirely above the openings 7 for the eyes; a chin region 12 comprising that portion entirely below the opening 11 for the mouth; and a mid region 10 that includes all other portions of the facial mask 1.

Note that the perimeter 3 is generally of a gently curving, primarily arcuate shape that generally outlines or conforms to the shape of a typical human face. In accordance with the invention, the perimeter 3 may also encompass various features in order to enhance the fit and comfort for users having a wide range of facial shapes and sizes.

Liquid-retaining layer 5 may include additional features to help facilitate good fit of the liquid-retaining layer 5 on a variety of facial shapes and sizes. For example, liquid-retaining layer 5 includes a pair of slits 9A, 9B in forehead region 8. Slits 9A, 9B result in the formation of a pair of laterally-extending tabs 13A, 13B. Facial mask 1 also includes a pair of slits 15A, 15B located in the chin region 12. Slits 15A, 15B result in the formation of a pair of laterally-extending tabs 17A, 17B.

In one embodiment, slits 9A, 9B are long enough that, in use, laterally-extending tabs 13A, 13B overlap one another. Similarly, slits 15A, 15B are long enough that, in use, laterally-extending tabs 17A, 17B overlap one another. The overlapping of the various tabs tends to provide an adjustable and comfortable fit as well as a strong tendency to stay-in-place on the user's face.

The function of the liquid-retaining layer is to absorb and retain liquids, e.g., a liquid impregnate, and hold it in contact with the user's facial skin. As such, the liquid-retaining layer comprises a liquid-retaining materials such as fibrous materials, natural sponges, synthetic sponges, or hydrogels.

In one embodiment of the invention, the liquid-retaining layer includes or consists essentially of a fibrous, non-woven material. As used herein, “fibrous, non-woven” means a material formed of fibers that are not woven into a fabric but rather are formed into a sheet, mat, or pad. The fibers can either be random (i.e., randomly aligned) or they can be carded (i.e., combed to be oriented in primarily one direction). Furthermore, the non-woven can be composed of a combination of layers of random and carded fibers. The fibers may be bonded to one another via physical entangling, thermal bonding, chemical bonding or similar means.

In order to enhance the durability of the liquid-retaining layer, the fibrous, non-woven material is formed such that it is not “paper” or “paper-like.” In this embodiment of the invention, more than about 50% of the fibrous mass of the fibrous, non-woven material is made of fibers having a length to diameter ratio greater than about 300. While the fibers may be staple fibers or continuous filaments, it is preferred that the fibers are staple fibers.

The fibrous, non-woven material may be comprised of a variety of natural and/or synthetic materials. By “natural” it is meant that the materials are derived from plants, animals, insects, or byproducts of plants, animals, and insects. By “synthetic” it is meant that the materials are obtained primarily from various man-made materials or from natural materials, which have been further altered. Non-limiting examples of natural materials useful in the present invention are silk fibers, keratin fibers (such as wool fibers, camel hair fibers) and cellulosic fibers (such as wood pulp fibers, cotton fibers, hemp fibers, jute fibers, and flax fibers).

Examples of synthetic materials include, but are not limited to, those selected from the group containing acetate fibers, acrylic fibers, cellulose ester fibers, cotton fibers, modacrylic fibers, polyamide fibers, polyester fibers, polyolefin fibers, polyvinyl alcohol fibers, rayon fibers, polyurethane foam, and mixtures thereof.

Synthetic materials, including fibrous, nonwoven materials made from one or more of the natural and synthetic materials useful in the present invention can be obtained from a wide variety of commercial sources such as Freudenberg & Co. (Durham, N.C. USA), BBA Nonwovens (Nashville, Tenn. USA), PGI Nonwovens (North Charleston, S.C. USA), Buckeye Technologies/Walkisoft (Memphis, Tenn. USA), Sansho Shigyo K. K. (Tosa City, Kouchi, Japan), and Fort James Corporation (Deerfield, Ill. USA).

Methods of making non-woven materials suitable for use in the liquid-retaining layer are also well known in the art. Such methods include, but are not limited to, air-laying, water-laying, melt-blowing, spin-bonding, or carding processes. The resulting non-woven material, regardless of its method of production or composition, is then generally subjected to at least one of several types of bonding operations to anchor the individual fibers together to form a self-sustaining web. The non-woven material can be bonded together by a variety of processes including hydro-entanglement, thermally bonding, chemical bonding and combinations of these processes. Moreover, the nonwoven may consist of a single layer or multiple layers. If the nonwoven is multiple layers, the layers are preferably bonded using the bonding processes described above.

Strength or firmness of the non-woven material may be a desirable attribute. This can be achieved, for example, by the addition of binding materials, such as wet strength resins, or the material may be made of polymer binder coatings, stable fibres, e.g. based on cotton, wool, linen and the like. Examples of wet strength resins include, but are not limited to, vinyl acetate-ethylene (VAE) and ethylene-vinyl chloride (EVCL) Airflex emulsions (Air Products, Lehigh, Pa.), Flexbond acrylic polymers (Air Products, Lehigh, Pa.), Rhoplex ST-954 acrylic binder (Rohm and Haas, Philadelphia, Pa.), and Ethylene-vinyl acetate (EVA) emulsion (DUR-O-SET® by National Starch Chemicals, Bridgewater, N.J.). The amount of binding material in the non-woven material may range from about 5% to about 20%, by weight, of the non-woven material. In one embodiment, the non-woven material is flushable, i.e., the non-woven material will pass through at least 10 feet of waste pipe in two toilet flushes. The material may also be biodegradable.

Non-woven materials of increased strength can also be obtained by using the so-called spunlace or hydro-entanglement technique. In this technique, the individual fibers are twisted together so that an acceptable strength or firmness is obtained without the need to use binding materials. The advantage of the latter technique is the excellent softness of the non-woven material.

In another embodiment, the liquid-retaining layer includes a hydrogel. By “hydrogel” it is meant a continuous network of polymer chains that are water-insoluble, sometimes found as a colloidal gel in which water is the dispersion medium, e.g., a multicomponent system consisting of a three-dimensional network of polymer chains and water that fills the space between macromolecules. The hydrogel may be a freestanding layer, e.g., may have sufficient mechanical integrity to be separately peeled-off, picked up or transported as a cohesive unit.

In one particularly preferred embodiment, the liquid-retaining layer is composed predominantly of a fibrous, non-woven material, a hydrogel, or combinations thereof; e.g., the liquid-retaining layer may be at least about 25% by weight (exclusive of any liquid impregnate) of such materials, more preferably at least about 50% by weight.

In one embodiment, the liquid-retaining layer includes a stretchable or elastic material that is capable of fully recovering after being placed under tension of 50% or 100% strain, such as may be included for use on the laterally-extending tabs or across the entire facial mask.

The basis weight of the liquid-retaining layer may range from about 10 grams per square meter (gsm) to about 200 gsm, such as between about 30 gsm and about 100 gsm. The liquid-retaining layer may have an average thickness that is less than about 5 mm, such as between about 0.1 mm and about 1 mm.

In one embodiment of the invention, the non-woven material may include a superabsorbent polymer. For the purposes of the present invention, the term “superabsorbent polymer” refers to materials, which are capable of absorbing and retaining at least about 10 times their weight in water under a 0.5 psi pressure. The superabsorbent polymer particles of the invention may be inorganic or organic crosslinked hydrophilic polymers, such as polyvinyl alcohols, polyethylene oxides, crosslinked starches, guar gum, xanthan gum, and other material known to the art of absorbent article manufacture.

Additives may also be added in order to increase the softness of the non-woven material. Examples of such additives include, but are not limited to, polyols such as glycerol, propylene glycol and polyethylene glycol, phthalate derivatives, citric esters, surfactants such as polyoxyethylene (20) sorbitan esters, and acetylated monoglycerides.

Sensory attributes may also be incorporated in the liquid-retaining layer. Examples of such sensory attributes include, but are not limited to color, texture, pattern, and embossing of the fibrous, non-woven material.

The liquid-retaining layer when laid flat, have a total surface area of about 100 cm² to about 1000 cm², such as from about 200 cm² to about 500 cm², such as between about 200 cm² to about 360 cm².

### Isolation Layer

The facial mask of the present invention comprises at least one isolation layer. As used herein, “isolation layer,” means a layer of material capable of reducing the areas of contact of the liquid-retaining layer with itself while the user is removing the liquid-retaining layer from its package, unfolding the liquid-retaining layer, or and placing it in contact with her face. The isolation layer may be discarded by the user after the user applies the liquid-retaining layer to her face. The isolation layer is preferably water-insoluble (as defined previously), or, at least insoluble in any impregnate which is present in the liquid-retaining layer 5.

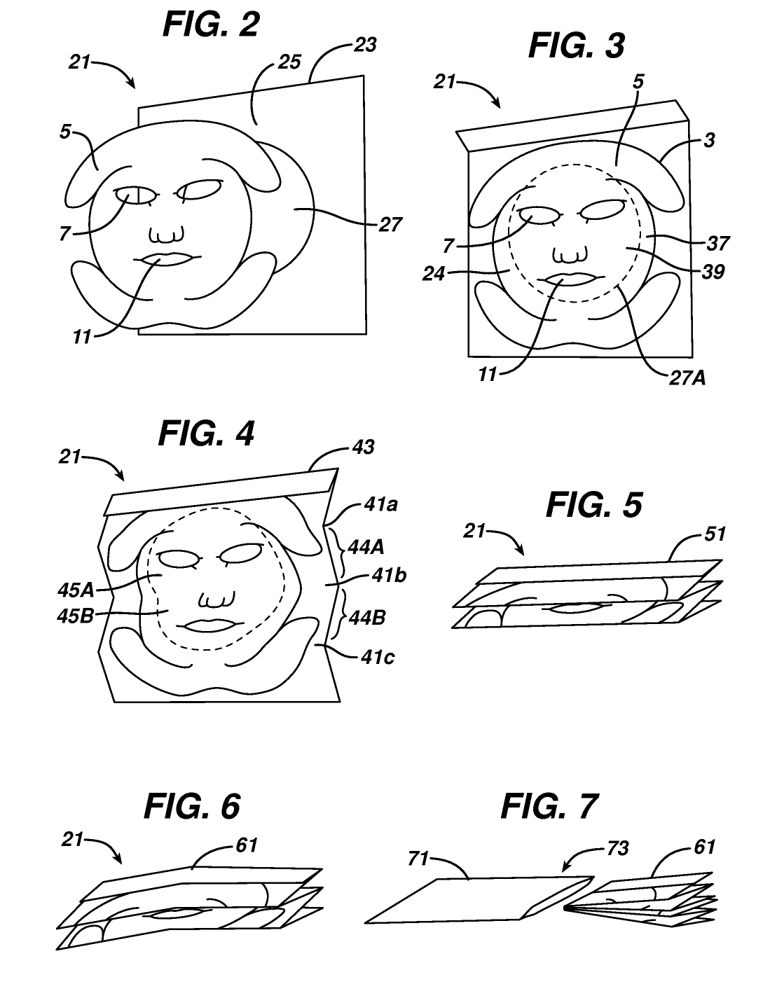

FIG. 2 depicts a facial mask consistent with embodiments of the invention described herein. Facial mask 21 includes the liquid-retaining layer 5 and an isolation layer 23. FIG. 2 depicts facial mask 21 in an unfolded state and, as such, both the liquid-retaining layer 5 and the isolation layer 23 exist, for example, as essentially flat/planar sheets.

The isolation layer 23 is readily separable from said liquid-retaining layer 5. As used herein, “readily separable” means capable of easy separation by hand using for example a gentle peeling motion. The isolation layer 23 is designed and/or formed from materials such that the liquid-retaining layer 5 (optionally comprising liquid impregnate) and the isolation layer 23 are readily separable from one another in one or both of the following situations: (1) when the facial mask 1 is unfolded and the liquid-retaining layer is in intimate contact with the isolation layer, and/or (2) when the facial mask is placed against the user's face with the liquid-retaining layer in contact with the face. In the latter case, the isolation layer is gently peeled from or otherwise separated from the liquid-retaining layer, leaving only the liquid retaining layer behind on the user's face. Isolation layer 23 is also preferably designed such that it is also readily separable from itself, e.g., particularly when the isolation layer is folded and in contact with itself. Easy separation of the isolation layer 23 from itself is particularly beneficial when the liquid-retaining layer includes a liquid impregnate, and even more so when the liquid impregnate is present in high loading levels, i.e., when the liquid retaining layer is wet.

Referring again to FIG. 2, the isolation layer 23 has a contact surface 25 for contacting at least portions of the liquid-retaining layer 5 and optionally an opposing surface (not shown in FIG. 2) on the reverse side of the isolating layer 23.

As shown in FIG. 3, when the liquid-retaining layer 5 and the isolation layer 23 are brought into contact with one another and made to substantially overlap, such as when the facial mask 1 is assembled and packaged, at least a portion of the liquid-retaining layer 5 overlaps a contact portion 24 of the isolation layer 23, forming an overlapping portion 37. The overlapping portion 37 corresponds with the area of the liquid-retaining layer 5 outside the phantom circular boundary 27A, (which is the boundary of opening 27 shown in FIG. 2.) but still within the perimeter 3 of the liquid-retaining layer 5. The area of the liquid-retaining layer 5 within the circular boundary 27A is an optional non-overlapping portion 39, i.e., one in which the liquid-retaining layer 5 does not contact the isolation layer 23.

According to the invention, as shown in FIG. 3, at least one and preferably all of the openings 7, 11 of the liquid-retaining layer 5 are unobstructed by the isolation layer 23. As used herein, “unobstructed” means at least about 80%, preferably 100%, of the area of such openings 7, 11 in the liquid-retaining layer 5 is or can be aligned with void (e.g., opening 27) in the isolation layer 23. The void or opening in the isolation layer is “continuous,” in that it consists entirely of empty space not comprising a mesh or other lattice of material.

In one particularly desirable embodiment, the size of opening in the isolation layer is greater than at least one opening in the liquid-retaining layer. That is, the opening in the isolation layer has a sufficient size such that at least one, preferably all, of the openings in the liquid-retaining layer fit well within the opening in the isolation layer. This alignment of the openings in the liquid-retaining layer and the isolation layer provide the user with comfort when placing the facial mask against her face, since the isolation layer is not positioned over or against her eyes or mouth, leaving them uncovered during application of the facial mask. Note the relative sizes and positioning of openings 7, 11, and 27 in FIG. 3.

The opening in the isolation layer also serves to prevent a “vacuum” effect in which air trapped between the isolation layer and the liquid-retaining layer might make it more difficult to separate the liquid-retaining layer and the isolation layer during application of the facial mask.

The area of the opening in the isolation layer is preferably at least as large as at least one opening in the liquid-retaining layer, e.g., at least about 5 cm². In one desirable embodiment, the opening in the isolation layer is nearly about as large as the surface area of a typical face, such as from about 100 cm² to about 325 cm².

In order to facilitate ease of separation of liquid-retaining layer 5 and the isolation layer 23, as shown in FIG. 4, the liquid-retaining layer 5 and the isolation layer 23 may be folded about one or more common fold axes. Three common fold axes 41a, 41b, 41c are shown in FIG. 4. The common fold axes 41a, 41b, 41c are shown as lines but may alternatively be curved. While FIG. 4 shows the common fold axes 41a, 41b, 41c as three parallel lines, various shapes and orientations are possible.

Also shown in FIG. 4 is an additional fold axis 43 about which the isolation layer 23 alone is folded. The additional fold axis 43 may be positioned so as to “trap” or “sandwich” a portion of the liquid-retaining layer 5 between portions of the isolation layer 23. For example, referring to both FIG. 4 and FIG. 1, additional fold axis 43 is positioned to overlap a portion of the forehead region 8, thus forming additional overlapping portions (and therefore additional contact surfaces 25) of the isolation layer 23 and the liquid-retaining layer 5.

It is preferred that the fold axes are disposed such that the portion of liquid-retaining layer that comes into contact with itself is minimized. However, by employing an isolation layer that does not obstruct the openings in the liquid-retaining layer, fractions of the liquid-retaining layer do contact one another upon folding. Referring to FIG. 4, two rectangularly-extending regions 44A, 44B of the facial mask are folded along fold lines 41A, 41B such that only the two rectangularly-extending regions 45A, 45B contact one another. All of the portions of the liquid-retaining layer 5 that are within each of the regions 44A and 44B, specifically fractions 45A and 45B, will contact one another (i.e., fraction 45A will be folded onto fraction 45B). Ideally, the portion of self-contact (i.e., the area of fraction 45A plus the area of fraction 45B divided by the total area of the top surface of the liquid-retaining layer 5) will be less than 90%, preferably less than 70%, more preferably less than 60%, of the total area of the liquid-retaining layer.

In order to minimize the portions of the liquid-retaining layer that contact one another, and additionally minimize the impact of this contact, in one desirable embodiment, the liquid-retaining layer 5 is folded in a “Z-fold” otherwise known as an “accordion-style” fold. As can be seen in FIG. 4, by folding along common fold axes in an accordion-style, the liquid-retaining layer 5 is much more easily separated from itself. Without being bound by theory, it is believed that this is because an accordion shape is easily placed in tension when unfolded, enhancing the force that pulls the layers apart upon unfolding.

The facial mask 1 folded about the common fold axes 41a, 41b, 41c and fold axis 43 results in a partially folded body 51, shown in FIG. 5. Specifically, the partially folded body 51 of FIG. 5 has an accordion shape. As shown in FIG. 6, the facial mask 21 may be folded about additional fold axes, e.g., perpendicular to fold axes 41A, 41B, 41C such as common fold axis 61 to form a folded body 61, as shown in FIG. 7. The folded body 61 may be placed inside a container 71 to provide a packaged product 73, suitable for placement into a larger package such another pouch or a box (not shown) and for sale to the consumer. Depending upon how the additional fold axes are oriented, direct contact of additional portions of the isolation layer 23 may be made with the liquid-retaining layer 5, and thereby create additional overlapping portions and therefore additional contact surfaces of the isolation layer 23.

Note that while, FIG. 2 depicts one opening 27 in the isolation layer 23, it is possible to include multiple openings in the isolation layer 23. Such openings in the isolation layer may separately and substantially correspond to the various openings in the liquid-retaining layer. For example, the isolation layer 23 may include a first opening that substantially corresponds with (i.e., is about the same dimensions as and overlaps) the mouth opening 11 in the liquid-retaining layer. A pair of second openings in the isolation layer may substantially correspond with eye openings 7 in the liquid-retaining layer. In this particular embodiment, although the benefit of no eye/mouth obstruction is achieved, the material costs are higher as compared with an isolation layer 23 having a single, large opening 27, as shown in FIG. 2. In addition, the manufacturing of multiple openings having more complex shapes and the need to register those openings with openings in the liquid-retaining layer adds to the cost of production.

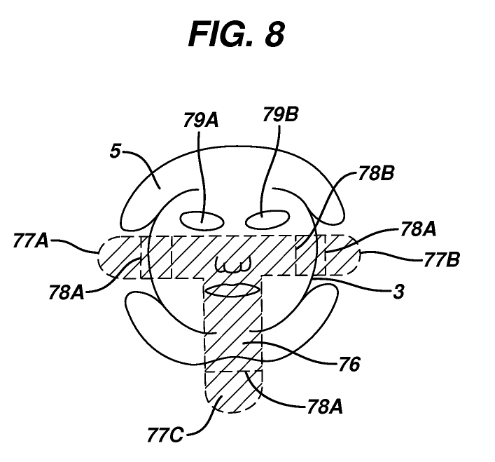

As shown in FIG. 8, in another embodiment, obstruction of openings 79A, B of the liquid-retaining layer may be reduced or eliminated by utilizing an isolation layer 76 that includes a plurality of protuberances 77A, 77B, 77C that extend beyond perimeter 3 of the liquid-retaining layer. The remainder of the isolation layer 76 is within the perimeter 3 of the liquid-retaining layer. The “T-shape” of isolation layer 76 is such that it does not comprise an internal opening such as opening 27 of FIG. 2. Rather, in this embodiment, isolation layer 76 does not adjoin the openings of the liquid-retaining layer. While the area of the isolation layer 76 may be reduced by making the protuberances 77A, 77B, 77C thinner, good separability of the isolation layer from the liquid-retaining layer can still be achieved if the protuberances extend beyond the perimeter 3 of the liquid-retaining layer and by folding the facial mask in an accordion-style, such as along common fold axes 78B and fold axes 78A.

In one embodiment, the contact surface 25 of the isolation layer 23 is hydrophobic or water-repellent (e.g., when laid flat, the contact surface 25 of the isolation layer can maintain a contact angle with a bead of deionzed water that is greater than about 20° for at least 5 seconds, more preferably at least about 40°, most preferably at least about 60°). For example, the isolation layer 23, in one preferred embodiment, includes or consists essentially of a hydrophobic film. By “film” it is generally meant a layer of material that is continuous over X and Y dimensions of at least about 100 microns. By “continuous” it is meant that the layer includes at least portions across its surface that are substantially devoid of capillary pores. By “capillary pores” it is meant pores of dimensions that are found in typical fibrous nowoven materials, such as those that could otherwise enhance capillary forces, capable of absorbing liquids and could cause undesirable sticking between the isolation layer and liquid-retaining layer. The capillary pores typically have a size from about 5 microns to about 200 microns and may be formed completely through the entire thickness dimension of the isolation layer. By substantially devoid, it is meant that less than about 50% of the film's contact surface includes capillary pores, preferably less than about 25%, more preferably less than about'5%.

In a preferred embodiment, the isolation layer may be a freestanding film such as a plastic film. The film may be formed by typical methods such as extrusion or similar methods known for producing thermoplastic films.

In one desirable embodiment, in order to enhance the ability to separate the isolation layer from the liquid-retaining layer, the isolation layer includes voids that are larger than the capillary voids described above and may be formed completely through a thickness of the isolation layer. For example, the isolation layer may include apertures (such as may be formed via water or hot air, pins, and the like) or holes that may have a diameter that is least about 200 microns (0.2 mm) to about 20 mm, preferably from about 400 microns to about 15 mm, such as from at least about 500 microns to about 10 mm. The basis weight of the apertured film of the isolation layer may be from about 15 gsm to about 50 gsm, such as from about 20 gsm to about 40 gsm, preferably from about 25 gsm to about 40 gsm. Furthermore, it may be desirable for the apertured film to comprise internally compounded surfactant (e.g., mono- and di-esters of various fatty acids or other similar chemistries such as ATMER, available from ICI Americas, Inc., Wilmington, Del.; AHCOVEL, available from Clariant.; MASIL, available from PPG Industries, Inc., Gurnee, Ill.; and MAPEG, available from PPG Industries, Inc.)

Apertures present in the isolation layer may be asymmetric about a plane through the thickness of the isolation layer. For example, the apertures may have sidewalls that protrude below a bottom plane of the isolation layer. Such an aperture geometry is typical of apertured films formed from hot air or water aperturing processes, such as those described in US Published Patent Application No. US20030171730 and U.S. Pat. No. 5,997,986. The side of such film that is in the direction downstream of the flux of the incident fluid used to form the apertures, i.e., the “rough” side of the apertured film, is also sometimes referred to as the “male” side. Conversely the side of the film that is upstream of the flux of the incident fluid, i.e., the “smooth” side of the apertured film, is also sometimes referred to as the “female” side. Either the rough side or the smooth side of the film is generally suitable for direct contact with the liquid-retaining layer.

In certain embodiments, the total contact area of all isolation layers in contact with the liquid-retaining layer when laid flat may be relatively small, as compared to the area of the liquid-retaining layer. As used herein, “total contact area of the isolation layer” means the total surface area of the isolation layer(s) in contact with the liquid-retaining layer when the facial mask is laid flat. If isolation layers contact both faces of the liquid-retaining layer, then the total contact area would include the area of contact on both faces of the liquid-retaining layer. The total contact area excludes the area of openings in the isolation layer, but would include any small apertures or other holes that are individually less than 1.0 mm in dimension. In certain embodiments, the total contact area of the isolation layer is less than 200% of the area of the liquid-retaining layer, preferably less than 150%, more preferably less than 100%, and, in certain embodiments, even less than 75% of the area of the liquid-retaining layer.

Suitable materials that may be included in the isolation layer include, for example, thermoplastics or thermoset polymers such as polyolefins (e.g., polyethylene, polypropylene), polyesters, polyurethanes, polytetraflyoroethyelene, polysilicones, and the like.

In another embodiment, the isolation layer comprises or consists essentially of a fibrous, nonwoven layer. In this embodiment, the isolation layer may be composed of only one type of fiber, such as polyester or polypropylene or it may include a mixture of more than one fiber. The isolation layer may be composed of bi-component or conjugate fibers having a low melting point component and a high melting point component. The fibers may be selected from a variety of natural and synthetic materials such as nylon, polyester, rayon (in combination with other fibers), cotton, acrylic fiber and the like and combinations thereof.

It is generally preferable that the isolation layer meet one or more of the following criteria: (1) it should not be so thin as to be difficult to peel using ones fingers, yet should not be so thick as to make the isolation layer too stiff or not sufficiently drapeable; and (2) in order to limit the adverse effect of capillary forces during separation of the isolation layer from the liquid-retaining layer, the isolation layer is preferably hydrophobic or water repellent. These criteria are particularly important if the contact surface of the isolation layer is a fibrous, nonwoven material.

If the isolation layer includes a fibrous, nonwoven layer, and particularly if the fibrous, non-woven layer of the isolation layer contacts the liquid retaining layer, it is preferred that the fibrous, nonwoven layer meet one of more of the following criteria: (1) a thickness (as measured using a footed dial thickness gauge with stand, available from B.C Ames of Melrose Massachusetts, with a 2″ diameter foot at a pressure of 0.07 prig and a readout accurate to 0.001″) of about 1 mm to about 5 mm, preferably from about 1.5 mm to about 4 mm; (2) a basis weight (mass per unit cross sectional area) in the range of about 10 gsm to about 75 gsm; (3) a flexural resistance (MCB) of less than 400 g, preferably less than 250 g and most preferably less than 150 g (MCB may be measured using a standard method, such as the method disclosed in US Published Patent Application No. US20080091157); or (4) include or consist essentially of fibers that are hydrophobic or have hydrophobic outer surfaces or sheaths; or have a hydrophobic coating formed on the fibrous, nonwoven. Furthermore, the nonwoven fibers may be chosen to be predominantly hydrophobic, i.e., should be predominantly formed from fibers that are hydrophobic or surface treated to render them hydrophobic.

Suitable hydrophobic fibers include polyolefins (polyethylene, polypropylene and combinations thereof) or polyesters that are free of hydrophilic finishes and coatings. Bi-component fibers such as those made up of a core such as polyester layer and a hydrophobic sheath such as one of polyethylene may also be suitable. In one embodiment, the percentage of fibers that are hydrophobic is at least about 50%, such as at least about 75%, preferably at least about 90%. The remainder of fibers may be hydrophilic fibers such as rayon or cellulose fibers, and fibers that would otherwise be hydrophobic, but are treated with a hydrophilic finish that renders the fibers wettable with deionized water.

Alternatively, the entire fibrous, non-woven isolation layer or strips or portions thereof may are coated with a hydrophobic material. The hydrophobic material may be a wax (vegetable, animal or mineral derived fatty esters, including glyceryl fatty esters) or other suitable hydrophobic compound, such as one that is solid or paste at room temperature. The coating may be formed to a thickness of about 0.25 mm to about 2 mm, across portions of the nonwoven, such as by slot coating or other similar coating methods that are suitable for forming a coating on a fibrous, nonwoven.

In another embodiment, the isolation layer includes a metal foil such as a thin layer of metal (e.g., aluminum) foil. In another embodiment, the isolation layer includes a substrate (e.g., a fibrous, nonwoven layer, a film, a foil, and the like) that has a hydrophobic coating or other surface modification to repel water. The hydrophobic coating or other surface modification may be a polymer and/or silicone resin that is coated upon and modifies the surface of a substrate of the isolation layer.

In a preferred embodiment, the entire isolation layer or the contact portion thereof should be liquid-impermeable. As used herein, “liquid-impermeable” means that when water is placed on top of the contact portion of the isolation layer (such as by placing a drop of deionized water applied via a pipette onto one half of a square centimeter of isolation layer), the water does not penetrate so as to wet or moisten the opposing surface of the contact portion of the isolation layer within a time period of about 120 minutes. In a preferred embodiment, at least about 25% of the total contact area of the isolation layer 23 is liquid-impermeable. In a further preferred embodiment, at least about 50% of the total contact area of the isolation layer 23 is liquid-impermeable. In an even further preferred embodiment, at least about 90% of the total contact area of the isolation layer 23 is liquid-impermeable.

While the isolation layer is shown as a square/rectangular shape, various other shapes for the isolation layer are contemplated. For example, the isolation layer may have a facial shape similar to the liquid-retaining layer, circular, oval, X-shaped, cross-shaped, bar-shaped, or any other suitable shape.

### Liquid Impregnate

The facial mask may include a liquid impregnate, such as may be used to moisten or wet the liquid-retaining layer. In one embodiment of the invention, the liquid impregnate is present in an amount sufficient to wet a user's facial skin when laid onto such skin. In another embodiment, the liquid impregnate is present in an amount sufficient to permit a first portion of the liquid-retaining layer to cohesively attach to a second portion of the liquid-retaining layer, wherein the first portion of the liquid-retaining layer is distal from the second portion of the liquid-retaining layer. Such cohesive attachment may be for a period of time of at least about 5 minutes when the liquid-retaining layer is laid on the face and the face is in an upright, vertical orientation.

In order to provide sufficient drapeability of the facial mask, the liquid impregnate may be present in an amount that is at least about 5% by weight of the weight of the liquid-retaining layer alone (i.e., the dry liquid-retaining layer). More preferably the liquid impregnate is present in an amount that is at least about 50%, even more preferably at least about 100%, even more preferably at least about 200%, such as from about 200% to about 300% by weight of the weight of the liquid-retaining layer. When employed in this amount, the liquid impregnate may be readily transferred to skin. To further enhance the transfer of the liquid impregnate to the skin of the user, such as for a hydrating facial mask, the liquid impregnate may be present in an amount greater than about 50% by weight, such as greater than about 65%, such as between about 65% to about 95%, by weight of the liquid-retaining layer.

The liquid impregnate may include an aqueous phase, an oily/hydrophobic phase, a gel phase, or a mixture of these phases. In one desirably embodiment, the liquid impregnate includes an aqueous phase, and even more preferably, the aqueous phase is an external phase (in which an oily phase or particulate phase may be dispersed, suspended or emulsified).

In one embodiment, the liquid impregnate has a viscosity that is less than about 10,000 centipoise (cps), when measured using a Brookfield digital viscometer, Model DV-II+Version 3.2 according to the operating instructions set forth in Manual No. M/92-161-H895, such as having a viscosity less than about 5000 cps, such as less than about 1000 cps. Such low viscosity liquid impregnates tend to be aesthetically pleasing to the user.

The liquid impregnate may include solvents such as water, including those that are humectants such as glycols including glycerin or propylene glycol, or alcohols such as isopropyl alcohol or ethanol. In one preferred embodiment, the liquid impregnate includes water, such as in a concentration of at least about 40%, more preferably at least about 60%, and even more preferably at least about 80% by weight of the liquid impregnate.

The liquid impregnate may include any of various ingredients known to the art of facial mask preparations, including: hydrophobic emollients such as fatty esters including esters of glycerin, fatty alcohols, hydrophobic polymeric emollients; sensory agents such as menthol and methyl lactate), chelating agents (such as EDTA), preservatives such as parabens, and other conventional cosmetic adjuvants, such as dyes, opacifiers (e.g., titanium dioxide and zinc oxide), pigments, fragrances, and microcapsules, such aminoplast microcapsules. One particular example of suitable microcapsules is polyoxymethyene melamine urea (PMU) microcapsules, commercially available as Pontenza Dimethicone from Reed-Pacific of Dural, Austrialia. Another such example is PMU Microcapsules (32 Micron Encapsulated Mineral Oil and Jojoba Oil), available from 3M Company of St. Paul, Minn.

Benefit Agents

In one embodiment of the invention, the liquid-retaining layer includes one or more benefit agents. What is meant by an “benefit agent” is a compound (e.g., a synthetic compound or a compound isolated from a natural source) that has a cosmetic or therapeutic effect on the skin including, but not limited to, lightening agents, darkening agents such as self-tanning agents, anti-acne agents, shine control agents, anti-microbial agents, anti-inflammatory agents, antifungals, anti-parasite agents, external analgesics, sunscreens, photoprotectors, antioxidants, keratolytic agents, moisturizers, nutrients, vitamins, energy enhancers, anti-perspiration agents, astringents, deodorants, hair growth inhibitors, anti hair-loss agents, hair growth promoters, hair removers, skin-firming agents, anti-callous agents, anti-aging agents such as anti-wrinkle agents, skin conditioning agents, allergy inhibitors, antiseptics, external analgesics, antipruritics, antihistamines, antiinfectives, anticholinergics, vasoconstrictors, vasodilators, wound-healing promoters, peptides, polypeptides, proteins, deodorants, anti-perspirants, film-forming polymers, counterirritants, enzymes, enzyme inhibitors, poison ivy treatment agents, poison oak treatment agent, burn treatment agents; anti-diaper rash treatment agents; prickly heat agents; herbal extracts; flavenoids; sensates; anti-oxidants, keratolytics; sunscreens; and anti-edema agents; and combinations thereof.

In one embodiment of the invention, the benefit agent is selected from, but not limited to, hydroxy acids, benzoyl peroxide, sulfur resorcinol, ascorbic acid and its derivatives, D-panthenol, hydroquinone, octyl methoxycinnimate, titanium dioxide, octyl salicylate, homosalate, avobenzone, polyphenolics, carotenoids, free radical scavengers, spin traps, retinoids such as retinol and retinyl palmitate, ceramides, polyunsaturated fatty acids, essential fatty acids, enzymes, enzyme inhibitors, minerals, hormones such as estrogens, steroids such as hydrocortisone, 2-dimethylaminoethanol, copper salts such as copper chloride, peptides containing copper, coenzyme Q10, lipoic acid, amino acids such a proline and tyrosine, lipo amino acids such as capryloyl glycine and sarcosine, vitamins, lactobionic acid, acetyl-coenzyme A, niacin, riboflavin, thiamin, ribose, electron transporters such as NADH and FADH2, and other botanical extracts, and salt, esters, and derivatives thereof. The benefit agent will typically be present in an amount of from about 0.001% to about 20% by weight of the liquid impregnate, e.g., about 0.01% to about 10% such as about 0.1% to about 5%.

Examples of vitamins include, but are not limited to, vitamin A, a vitamin B such as vitamin B3, vitamin B5, and vitamin B12, vitamin C, vitamin K, and vitamin E, and salts, esters, and derivatives thereof. (e.g., retinyl palmitate, ascorbyl acetate, and tocopherol acetate).

Examples of hydroxy acids include, but are not limited, to glycolic acid, lactic acid, malic acid, salicylic acid, citric acid, and tartaric acid.

Examples of antioxidants include, but are not limited to, water-soluble antioxidants such as sulfhydryl compounds and their derivatives (e.g., sodium metabisulfite and N-acetyl-cysteine), lipoic acid and dihydrolipoic acid, resveratrol, lactoferrin, and ascorbic acid and ascorbic acid derivatives (e.g., ascorbic acid glucoside, magnesium ascorbyl phosphate, and ascorbyl palmitate and ascorbyl polypeptide). Oil-soluble antioxidants suitable for use in the compositions of this invention include, but are not limited to, butylated hydroxytoluene, retinoids (e.g., retinol and retinyl palmitate), tocopherols (e.g., tocopherol acetate), tocotrienols, and ubiquinone. Natural extracts containing antioxidants suitable for use in the compositions of this invention, include, but not limited to, extracts containing flavonoids and isofavonoids and their derivatives (e.g., genistein and diadzein), extracts containing resveratrol and the like. Examples of such natural extracts include grape seed, green tea, pine bark, and propolis.

Examples of botanical extracts include, but are not limited to legumes such as Soy, Aloe Vera, Feverfew, Hedychium, Rhubarb, Portulaca, Cedar Tree, Cinnamon, Witch Hazel, Dandelion, Chinese Angelica, Turmeric, Ginger, Burnet, Houttuynia, Coix Seed, and Thyme. What is meant by a “botanical extract” is a blend of two or more compounds isolated from a plant.

In one embodiment of the invention, the benefit agent is designed for application on the forehead region and includes, but is not limited to: oil-control agents such as titanium dioxides, alcohols, botanical extracts, and talc; pore refining agents such as alpha-hydroxy acids, beta-hydroxy acids, and enzymes; anti-acne agents such as benzoyl peroxide, salicylic acid, trichlorcarban, triclosan, azelaic acid, clindamycin, adapalene, erythromycin, sodium sulfacetamide, retinoic acid, and sulfur; oil-absorbing agents such as titanium dioxides and clays; shine control agents such as silicones, alcohols, talc, and clays; dark spot reduction agents such as vitamin C, hydroquinone, botanical extracts, alpha-hydroxy acids, beta-hydroxy acids, and retinoids; and/or wrinkle/fine-line reduction agents such as retinoids, alpha-hydroxy acids, and enzymes.

In another embodiment of the invention, the benefit agent is designed for application around the mouth and includes, but is not limited to: hydration/moisturization agents such a glycerin, silicone, glycols, botanical extracts, and esters; pore-refining agents; anti-acne agents; vasodilators such as niacinamide and horsechesnut extract; vasoconstrictors such as caffeine and botanical extracts; skin-lifting agents such as (e.g., copper containing peptides, dimethyaminoethanol, and polymers); skin-firming polymers; wrinkle/fine-line reduction agents; depigmenting/skin lightening agents such as vitamin C, hydroquinone, botanical extracts, alpha-hydroxy acids, beta-hydroxy acids, retinoids, arbutin, and kojic acid; and depilatory/hair reducing agents such as soy extracts, n-acetyl-cysteine, and isoflavones.

While various combinations are contemplated, under one non-limiting example, one or more benefit agents are selected from the group consisting of ascorbic acid and its derivatives, alpha-hydroxy-acids, beta-hydroxyacids, alkanolamines, proteins, enzymes, and enzyme activators, and combinations thereof are in the liquid impregnate, and one or more benefit agents are selected from the group consisting of retinoids, tocopherols, enzymes, enzyme activators, and combinations thereof are within the liquid core.

In one embodiment of the invention, the product comprises an enzyme such as a lignin peroxidase and a suitable activator such as a peroxide (e.g., hydrogen peroxide) as described in WO 2004/052275.

The liquid impregnate may have a pH that is suitable for extended contact with the skin, such as from about 4.0 to about 8.0, more preferably from about 6.0 to about 7.5.

Packaging of Product

In one embodiment of the invention, the facial mask is in finished packaged form inside a package. In one embodiment, the container or package is formed from a plastic, metal or glass tube, tub, pouch or jar containing the facial mask. The product may further contain additional packaging such as a plastic or cardboard box for storing one or more of such containers (e.g., a package of two to twenty individual products). Non-limiting examples of material that may be used to manufacture such containers include aluminum, polypropylene, polyethylene, and/or polyesters. In one embodiment of the invention, the package is substantially air-impermeable.

In one embodiment of the invention, the product includes instructions directing the user to apply the facial mask to the skin, such as to the face. In one embodiment, where the liquid-retaining layer contains a liquid impregnate that is present in an amount at least about 5% by weight of the liquid-retaining layer, the instructions direct the use to apply the product directly to the skin. In another embodiment where the liquid-retaining layer contains a liquid impregnate that is present in an amount at least about 5% by weight of the weight of the liquid-retaining layer or a product that does not contain any liquid impregnate, the instructions direct the use to apply a liquid to the facial mask prior to application to the skin (e.g. to add water, a toner, or a cleanser to the facial mask).

In one embodiment, the instructions direct the user to apply the facial mask for the benefit of changing the appearance of the tone and/or color of the skin.

The instructions may direct contacting of the facial mask with the skin (e.g., the face) for a period of time, such as from about 10 seconds to about 1 hour (e.g., such as from about 1 minute to about 15 minutes). The user may also be directed to massage any liquid remaining on the skin after removal of the liquid-retaining layer. Such massaging may facilitate imparting improved color/tone uniformity in the skin of the subject.

Method of Making and Using the Product

Facial masks of the present invention may be made by various conventional methods, known to those skilled in the art. For example, a liquid-retaining layer, such as a sheet of non-woven material optionally perforated or cut to a pre-determined size to form a “blank,” of a size and shape suitable to fit over a human face, may be formed by methods already discussed. Openings may optionally be cut out of the blank corresponding to the eyes, nose, and/or mouth. Using a “subtractive” method one or more separation features such as slits, notches, and wedges may be sliced, cut or punched out of the blank. In one embodiment, the separation feature is formed by weakening or perforating the liquid-retaining layer rather than by slitting or removing material. Consistent with these embodiments, the facial mask may be of an “integrated” structure, in that the entire facial mask is essentially uniform throughout its area, as viewed from the top in a plan view.

In an alternative embodiment, the facial mask may be at least partially formed by an “additive” process, i.e. portions of the mask are stitched, bonded, or adhered together to create the separation features. After forming the liquid-retaining layer, the liquid-retaining layer may then be co-folded with the isolation layer and placed in a plastic pouch housing or other suitable container.

It is generally preferred that facial masks of the present invention are unitary, i.e., a one-piece mask that can be readily picked up with a user's hands and transported as single cohesive unit generally adapted to cover substantially the entire face of a user. However, the facial mask may also be formed from two or more pieces. Suitable two-piece mask designs with slits include a top piece that includes most or all of the forehead portion and a bottom piece that includes most or all of the chin portion. For embodiments in which there are two pieces, in order to determine the geometric centroid, angle of disposition, etc., the two pieces are placed in relative positions with respect to one another that are suitable for use.

The optional liquid impregnate may be prepared by mixing ingredients such as water and one or other ingredients and/or more benefit agents together to form a uniform liquid. The resulting liquid impregnate may then be poured into the package. Alternatively, the impregnate may be sprayed or otherwise distributed about the liquid-retaining layer.

The resulting facial mask may be individually sealed in the package or placed along with other liquid-retaining layers together into a single package. Multiple packaged liquid-retaining layers may be grouped together in an outer container, such as a box.

The user may unfold the facial mask (unfold the isolation layer and the liquid-retaining layer simultaneously), and place or position the mask such that the liquid-retaining layer intimately against the facial skin so as to cover substantially the entire face. The user may then peel off the isolation layer. The user may lie down or walk around, allowing the facial mask to treat the skin.

The facial mask achieves all the user comfort of a conventional facial mask with unobstructed openings for the eyes and or mouth. Yet, the facial mask may be much more readily unfolded than a conventional mask. In addition, these benefits are achievable with less material and cost than would be associated with a facial mask with two separate layers completely “sandwiching” the liquid retaining layer. Furthermore, because the facial mask of the present invention is easy to unfold, one can provide the mask to consumers in a highly folded state, thereby saving shelf or packaging space, without inconveniencing the consumer with a difficult unfolding process. The inventors have also surprisingly found that it is also possible to employ a range of materials in the isolation layer, yet still achieve this surprising combination of benefits.

The following example illustrates details of embodiments of the invention, without limiting it in any matter.

### EXAMPLES

A series of facial masks according to the invention were made using different isolation layers and tested as follows.

A liquid-retaining layer formed from a sheet of nonwoven fibers, (KP9560, a blend of 35% rayon and 45% pulp and 10% PET, 60 grams per square meter, commercially available from Sansho Shigyo K.K. of Tosa City, Kouchi, Japan) was cut to size a shape to fit a human face. The outer dimensions were about 20.3 cm×23.2 cm. Openings were cut out of the sheet corresponding to the eyes, nose, and mouth. The liquid-retaining layer had a design similar to that shown in FIG. 1. A liquid impregnate was prepared similar to the liquid impregnate used in commercially available NEUTROGENA Fine Fairness Mask with Vitamin C, commercially available from Neutrogena Corporation, Los Angeles, Calif.

Different isolation layers were prepared. They are described in TABLE 1. The various isolation layers were combined with the above liquid-retaining layer to form facial masks and subjected to three tests: (1) the “Folding/Unfolding Evaluation”; (2) the “Facial Evaluation;” and (3) “Release Test.”

The “Folding/Unfolding Evaluation” was conducted by providing the liquid-retaining layer impregnated with liquid as described above. The isolation layer to be tested was cut to include a rounded triangle-shaped hole in order to expose the openings in the liquid-release layer. The isolation layer was placed in intimate contact with the liquid-retaining layer, and then folded using fold lines as shown in FIG. 4-6. The isolation layer was assessed for the effort required to fold it together with the liquid-retaining layer, to unfold it, and whether it unadvantageously stuck to itself.

The “Facial Evaluation” was conducted by again providing the liquid-retaining layer impregnated with liquid. The isolation layer to be tested was cut as described above and again placed in intimate contact with the liquid-retaining layer. The layers were then picked up by a user and the liquid-retaining layer was placed against the face and the isolation layer was peeled from the liquid-retaining layer. The isolation layer was assessed for its ability to separate easily, but in a controlled fashion, from the liquid-retaining layer.

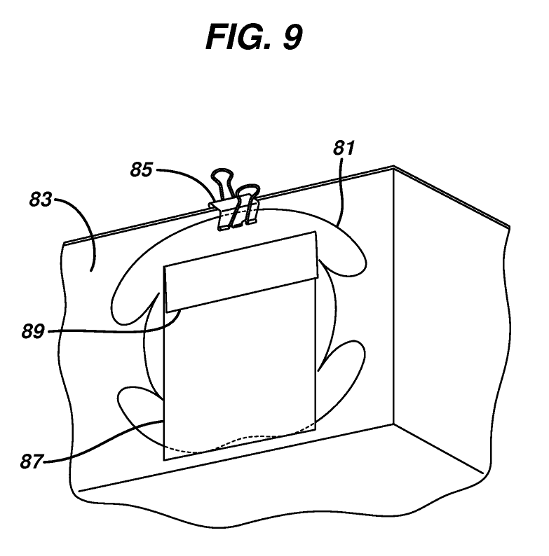

The “Release Test” was conducted according to the following method: 250 grams of the liquid impregnate was placed in a pan with dimensions (LWH) 27.9×17.8×3.8 cm (11″×7″×1.5″). The liquid-retaining layer was saturated with the impregnate by laying it in the pan and allowing it to sit for 30 seconds to hydrate. After saturating the liquid-retaining layer for 30 seconds, the excess impregnate was allowed to run off for 10 seconds by holding the saturated liquid-retaining layer vertically over the pan. The isolation layer to be tested was cut to a length of 23 cm (9.1″), which was the length of the mask used in the study. The width of the film was cut to a length of 17.5 cm (6.9″). Four staples (Nt. total Wt.≈0.10 grams), separated by 2.5 cm (1.0″) and 0.4″ (1 cm) down from the top border to provide a counterweight for the film were stapled across the top edge of the film. As shown in FIG. 9, hydrated liquid-retaining layer 81 was placed against a flat, vertical surface 83, such as the vertical wall of a tub or plastic basin. It was secured in positions using a 1″ binder clip 85.

Isolation layer 87 was then placed against the mask such that it was center justified and the bottom edges of the film and the mask were substantially aligned. The top edge 89 of the isolation layer 87 was folded down 4.5 cm (18″), as shown in FIG. 9. Once the fold was made, the time until the isolation layer separated entirely from the mask was measured using a Cole-Parmer® Model #94410-10 stopwatch. Three replicates were run and the average was taken. The trials were allowed to stand for a maximum of 30 minutes (1800 seconds).

TABLE 1

| | | | | Release | |
|---|---|---|---|---|---|
| | | Folding/ | | Test, | Release |
| | Isolation Layer | Unfolding | | Female | Test, Male |
| Ref. | Material | Evaluation | Facial Evaluation | (seconds) | (seconds) |
| Ex. 1 | Apertured film. | Good. Either | NOT TESTED | 18 | 1 |
| | 22 holes/inch; 23 | orientation easy to | | | |
| | gsm | fold and unfold. | | | |
| Ex. 2 | Apertured film. | Good. Either | Good. Either | 16 | 5 |
| | 40 holes/inch; 35 | orientation easy to | orientation easy to | | |
| | gsm | fold and unfold. | control unpeeling from | | |
| | | Creases well. | liquid retaining layer | | |
| | | | (liquid-retaining layer). | | |
| Ex. 3 | Apertured film. | Fair. Some | Fair. Peeling relatively | >1800 | >1800 |
| | No internal | problems with film | easy to control, but peel | | |
| | surfactant; 40 | sticking to itself; | resistance is somewhat | | |
| | holes/inch; 23 | especially in | high, especially for | | |
| | gsm | female orientation. | male side. | | |
| Ex. 4 | Wax-coated paper | NOT TESTED | NOT TESTED | >1800 | >1800 |
| Ex. 6 | Apertured film; | NOT TESTED | NOT TESTED | >1800 | >1800 |
| | 40 holes/inch; 23 | | | | |
| | gsm | | | | |
| Ex. 8 | Apertured film; | NOT TESTED | NOT TESTED | 5 | 3 |
| | 22 holes/inch; 35 | | | | |
| | gsm | | | | |
| Ex. 9 | Apertured film; | NOT TESTED | NOT TESTED | >1800 | >1800 |
| | 40 holes/inch; 23 | | | | |
| | gsm | | | | |
| Ex. 10 | Apertured film; | NOT TESTED | NOT TESTED | 27 | 5 |
| | 40 holes/inch; 23 | | | | |
| | gsm | | | | |
| Ex. 11 | Non-woven fabric | Moderate. | Good. Conforms well to | 16 | 15 |
| | coated with wax | Separates well | face and unpeels well. | | |
| | | from liquid- | | | |
| | | retaining layer, but | | | |
| | | sticks to itself a | | | |
| | | little bit more than | | | |
| | | optimal. Tabs on | | | |
| | | liquid-retaining | | | |
| | | layer don't stick | | | |
| | | well to this | | | |
| | | nonwoven. | | | |
| Ex. 12 | Through air- | Poor. Does not fold | Poor. Does not conform | 73 | 2 |
| | bonded nonwoven | well - too stiff. | to face - too stiff. | (smooth side) | (rough side) |

*All apertured film materials were “A-B-A” sandwich structures of low density polyethylene (LDPE) and linear low density polyethylene (LLDPE), with an open area 20-30%; compounded with titanium dioxide, and, except where indicated also compounded with an internal surfactant.

Surprisingly, a variety of effective isolation layer materials can be used with a liquid-retaining layer that is made from a fibrous, nonwoven material according to the invention. A wide variety of apertured film materials are suitable for use as the isolation layer, especially those with internally compounded surfactant. The inventors have also found that it is also even possible to use a fibrous, nonwoven material as the isolation layer, including nonwovens having a hydrophobic coating formed on portions of the isolation layer.

It is understood that while the invention has been described in conjunction with the detailed description thereof, that the foregoing description is intended to illustrate and not limit the scope of the invention, which is defined by the scope of the appended claims. Other aspects, advantages, and modifications are within the claims.

1. A facial mask, comprising:
a water-insoluble, liquid-retaining layer sized and shaped to lie against and substantially coincident with a face of a human user, wherein the water-insoluble, liquid-retaining layer comprises at least one opening formed therethrough, and
an isolation layer substantially overlapping and readily separable from said water-insoluble, liquid-retaining layer, wherein the opening in said water-insoluble, liquid-retaining layer is unobstructed by said isolation layer.

2. The facial mask of claim 1, wherein said isolation layer comprises at least one opening formed therethrough and aligned with the opening in said water-insoluble liquid-release layer.

3. The facial mask of claim 2, wherein the size of the opening in said isolation layer is greater than the size of at least one opening in said water-insoluble, liquid-retaining layer.

4. The facial mask of claim 1, wherein said water-insoluble, liquid-retaining layer and said isolation layer are folded about at least one common fold axis.

5. The facial mask of claim 1, wherein said water-insoluble, liquid-retaining layer and said isolation layer are folded about at least two common fold axes, such that the facial mask assumes an accordion shape.

6. The facial mask of claim 1, wherein the total contact area of said isolation layer is less than about 150% of the total area of said water-insoluble, liquid-retaining layer.

7. The facial mask of claim 1, wherein the total contact area of said isolation layer is less than about 100% the total area of said water-insoluble, liquid-retaining layer.

8. The facial mask of claim 1, wherein said water-insoluble, liquid-retaining layer comprises a fibrous, non-woven material.

9. The facial mask of claim 1, wherein said isolation layer is water-insoluble.

10. The facial mask of claim 1, wherein said isolation layer comprises a contact surface in contact with said water-insoluble, liquid-retaining layer, wherein said contact surface is hydrophobic.

11. The facial mask of claim 1, wherein said isolation layer comprises a plurality of apertures each having a diameter of about 200 microns to about 20 millimeters.

12. The facial mask of claim 1, wherein said isolation layer comprises a plurality of apertures each having a diameter of about 500 microns to about 10 millimeters.

13. The facial mask of claim 1, wherein said isolation layer comprises a contact surface in contact with said water-insoluble, liquid-retaining layer, wherein said contact surface comprises a fibrous, nonwoven material.

14. The facial mask of claim 13, wherein said fibrous, nonwoven material comprises fibers that are at least about 50% hydrophobic.

15. The facial mask of claim 1, wherein said isolation layer comprises an apertured thermoplastic film.

16. The facial mask of claim 1, wherein said isolation layer is adapted to extend beyond the perimeter of said water-insoluble, liquid-retaining layer when said water-insoluble, liquid-retaining layer is unfolded.

17. The facial mask of claim 1, further comprising a liquid impregnate absorbed onto said water-insoluble, liquid-retaining layer.

18. The facial mask of claim 17, wherein said liquid impregnate is present in an amount that is at least about 50% by weight of said water-insoluble, liquid-retaining layer.

19. The facial mask of claim 1, wherein the isolation layer comprises a plurality of protuberances that extend beyond the perimeter of the water-insoluble, liquid-retaining layer.

20. The facial mask of claim 1, wherein the isolation layer comprises at least one opening substantially coincident with at least one opening in the water-insoluble, liquid-retaining layer.
